(19) United States
(12) Patent Application Publication
(10) Pub. No.: US 2008/0221679 A1
(43) Pub. Date: Sep. 11, 2008

(54) Collapse-Resistant Breast Implant With Stiffened Walls

(75) Inventor: Robert S. Hamas, Dallas, TX (US)

Correspondence Address: SCOTT LADERE, 3001 10TH STREET, WAYLAND, MI, 49348, US

(73) Assignee: IDEAL IMPLANT INCORPORATED, Dallas, TX (US)

(21) Appl. No.: 12/043,549

(22) Filed: Mar. 6, 2008

Related U.S. Application Data
(60) Provisional application No. 60/905,241, filed on Mar. 6, 2007.

(51) Int. Cl. A61F 2/12

(52) U.S. Cl. 623/8

(57) ABSTRACT

A surgically-implantable breast prosthesis comprises an enclosing shell having an exterior surface and enclosing a lumen, the exterior surface defining an edge portion separating front and rear portions of the enclosing shell, wherein the lumen enclosed by the shell is able to accommodate a fluid, and the front rear portions of the enclosing shell are reinforced, thickened or stiffened on a central or front and rear surface spaced from the edge portion.

### BACKGROUND OF THE INVENTION

1. Field of the Invention

This invention relates generally to surgically-implantable mammary prostheses.

2. Description of Related Art

Breast prostheses have long been used for breast augmentation and for reconstructive surgery following a mastectomy. The prostheses are available in numerous sizes and shapes including teardrop, round, low profile, and high profile. Usually, breast prostheses are implanted via a small inframammary or peri-aerolar incision into a pocket dissected deep into the patient's own breast tissue in front of the pectoral muscle. In certain situations, the prosthesis may be placed behind the various chest muscles.

U.S. Pat. No. 5,480,430 entitled “Shape-Retaining Shell for a Fluid Filled Prosthesis” discloses a prosthesis shell that asymmetrically varies in thickness and, in particular, the walls and edge of the upper portion of the shell are thickened so as to prevent collapsing of the shell near the uppermost edge. In the examples disclosed in that patent, the prosthesis itself has an asymmetric outer configuration and the proper orientation at the time of implanting is readily apparent. Many breast prostheses have a symmetrical outer configuration. It would require extra precautions to recognize the portion of the prosthesis having the thicker wall and edge that must be oriented in the superior position at the time of implanting. Moreover, rotation of the prosthesis after implanting is likely unless prevented by various surgical means.

It is an advantage, according to the present invention, to provide an implantable breast prosthesis that by thickening and/or stiffening of a central portion of a wall of the shell, it will have improved resistance to collapsing near the top and resistance to sagging when the patient (implant) is upright.

### SUMMARY OF THE INVENTION

Briefly, according to the present invention, there is provided a surgically-implantable prosthetic device comprising a shell enclosing a lumen able to accommodate a fluid therein. The exterior surface of the outer shell defines an edge portion of the shell as viewed from a front direction. The edge portion separates front and rear portions of the outer shell. The prosthetic device further may comprise an inner shell able to accommodate a fluid and having an exterior surface that defines an edge portion of the inner shell that separates front and rear portions. The edge portions are not thickened and will be soft to the touch making it less likely to be felt through skin.

Both the outer lumen of the outer shell and the inner lumen of the inner shell may be filled with a fluid. A saline solution would be an appropriate choice for use as the fluid. Saline refers to any electrolyte combination together with water, however, the invention is not limited solely to the use of saline. Other fluids may be utilized, such as, for example, silicone gel, organic polymers or protein fluids; furthermore, certain gases may possibly be utilized as substitutes for fluids. Lubricating agents may be added to the saline. The outer lumen and/or the inner lumen may be pre-filled prior to implantation or, alternatively, may be first implanted and then filled with the fluid. One or more valves may be provided for the filling of the outer lumen or inner lumen.

It is an improvement, according to the present invention, that the front surface or front and rear surfaces of the outer shell and/or inner shell have thickened or stiffened central surfaces spaced from the edge portion of the respective shells.

These and other advantages of the present invention will be understood from the description of the desirable embodiments, taken with the accompanying drawings, wherein like reference numerals represent like elements throughout.

### BRIEF DESCRIPTION OF THE DRAWINGS

FIG. 1 is a schematic section view of a double lumen implant, according to the present invention, having thickened or stiffened front and back surfaces of the outer shell;

FIG. 2 is a front view of an implant, according to the present invention;

FIG. 3 is a schematic section view of a single lumen implant, according to the present invention, having thickened or stiffened front and back surfaces;

FIG. 4 is a schematic section view of a single lumen implant, according to the present invention, with a thickened or stiffened front surface;

FIG. 5 is a schematic section view of a double lumen implant, according to the present invention, with thickened or stiffened front and back surfaces of the inner shell; and

FIG. 6 is a schematic second view of a double lumen implant, according to the present invention, with thickened or stiffened front surfaces of the inner and outer shells.

### DESCRIPTION OF THE PREFERRED EMBODIMENTS

For purposes of the description hereinafter, the spatial or directional terms, such as “inner,” “outer,” “top,” “bottom,” “central,” and derivatives thereof, shall relate to the invention as it is oriented in the drawing figures. The “rear portion” of a breast implant is that intended to be adjacent the chest wall and the “front portion” is that portion facing away from the chest wall. However, it is to be understood that the present invention may assume various alternative variations, except where expressly specified to the contrary. It is also to be understood that the specific apparatus illustrated in the attached drawings and described in the following specification is simply an exemplary embodiment of the present invention. Hence, specific dimensions and other physical characteristics related to the embodiments disclosed herein are not to be considered as limiting.

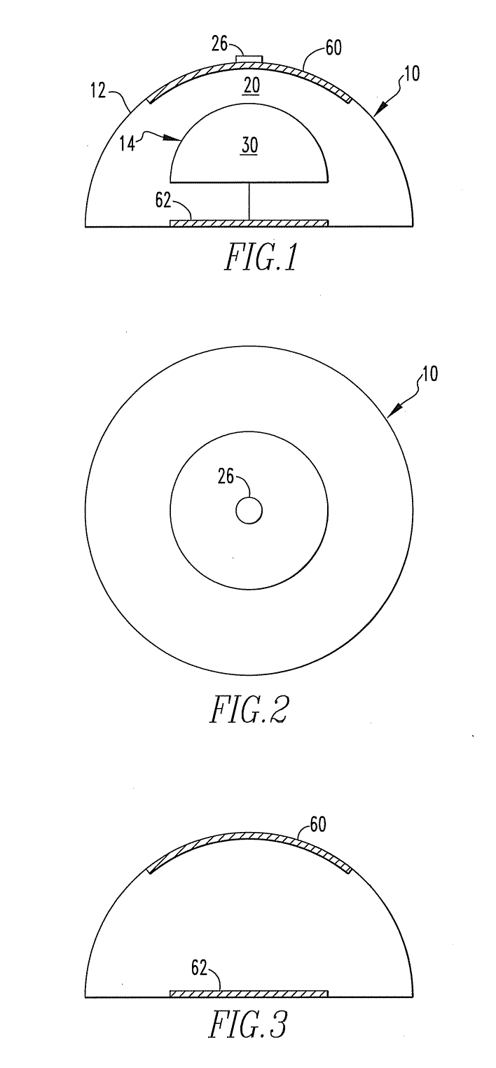

Implant 10 is illustrated in FIG. 1. The implant 10 is particularly adapted for use as a surgically-implantable mammary prosthesis. The implant 10 includes an outer shell 12 enclosing a lumen or a cavity 20, and an inner shell 14 enclosing a lumen or a cavity 30.

With reference to FIG. 1, the outer shell 12 defines an outer lumen 20 and includes an exterior surface and an interior surface. The outer shell 12 may include a valve 26 that bridges a portion between the exterior surface and the interior surface of the outer shell 12. The valve 26 may be placed along various areas of the outer shell 12. The valve 26 allows for filling of the outer lumen 20 of the outer shell 12 with a fluid after the manufacture of the implant 10, either before or after implantation into a patient. The fluid is preferably a saline solution, yet it is to be understood that the term fluid may refer to both gaseous and liquid fillers or any combination thereof including, but not limited to, electrolyte and organic solutions including polymers, such as silicone gel. The valve 26 also allows for the controlled removal of the fluid without damaging or destroying the implant 10. Alternatively, the outer lumen 20 may be manufactured as a pre-filled and completely sealed member (not shown) and, therefore, not require a valve 26 for the outer lumen 20.

The outer shell 12 is preferably constructed of a non-porous, flexible, biocompatible material, such as silicone elastomer. The outer shell 12 has a wall of sufficient thickness to provide structural integrity to retain fluids while achieving the desired flexibility and malleability of the implant 10. The outer shell 12 is substantially oval-shaped as viewed from the side and a generally circular shape as viewed from the front. Thus, the shape of the implant 10 is defined by the overall external shape of the outer shell 12. In an exemplary embodiment, the enclosed volume within the outer shell 12 is 375 cc. Therefore, the outer shell 12 may accommodate say 375 cc of volume-displacing material, e.g., flexible filler. It is to be understood that various other volumes of shells 12 may be utilized.

The inner shell 14 defines an inner lumen 30, and includes an exterior surface and an interior surface. The inner shell 14 is smaller than the outer shell 12 in that the diameter measurement and/or the projection measurement is less than that of the implant 10. In an exemplary embodiment, the enclosed volume of the inner shell 14 is 250 cc. The inner shell 14 is situated within the outer lumen 20 of the outer shell 12 in a relatively central position with respect to the interior surface of the outer lumen 20. Similar to the outer shell 12, the inner shell 14 may include a valve. The valve bridges the exterior surface and the interior surface of the inner shell 14, as well as the exterior surface and the interior surface of the outer shell 12. The valve (not shown) allows for filling of the inner lumen 30 of the inner shell 14 with the fluid after the manufacture of the implant 10, either before or after implantation into a patient. The valve also allows for the controlled removal of fluid without damaging or destroying the implant 10. Alternatively, the inner lumen 30 may be manufactured as a pre-filled and completely sealed member and, therefore, not requiring the valve.

Returning to FIG. 1, once implanted, the top or front of the implant 10 faces away from the chest wall of a patient.

The diameter measurement and the projection measurement of the outer shell 12 and the inner shell 14 may vary in differently sized alternative embodiments. These alternative embodiments may represent various sizes, shapes, or proportions of implants available to the patient. Additionally, the outer shell 12, the inner shell 14, both the outer shell 12 and the inner shell 14, or neither the outer shell 12 nor the inner shell 14 may be pre-filled with the fluid by a manufacturer of the implant 10. Thus, shells that are not pre-filled require the valve to be incorporated therein.

Referring to FIGS. 1 and 2, the front and back portions of the outer shell 12 are thickened in the central portions 60 and 62. Although it is not completely understood, it has been found that the thickening or stiffening of the central portions of the outer shell assists in preventing the sagging and collapse of the peripheral portions of the implant. Moreover, the non-thickened edge portions retain flexibility so that the implant is softer to the touch and less likely to be felt through the skin.

Referring to FIG. 3, there is shown an embodiment of a single lumen implant in which the front and back surfaces 60, 62 are thickened or stiffened.

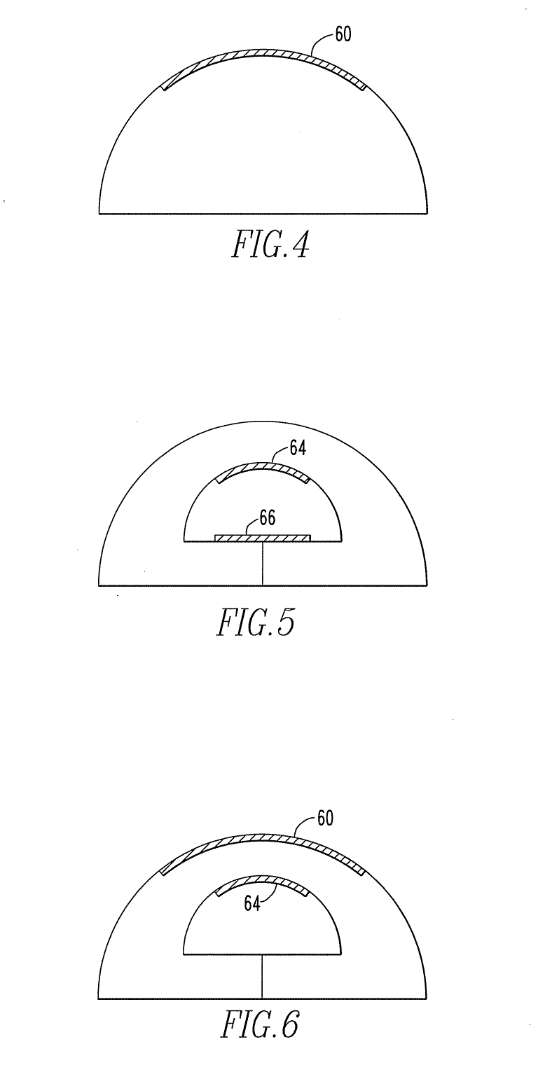

Referring to FIG. 4, there is shown an embodiment of a single lumen implant in which the front surface 60 is thickened or stiffened.

Referring to FIG. 5, there is shown an embodiment of a double lumen implant with thickened or stiffened front and back surfaces 64, 66 of the inner shell enclosing the inner lumen.

Referring to FIG. 6, there is shown an embodiment of a double lumen implant with thickened or stiffened front surfaces 60, 64 of the outer and inner shells.

Having thus described my invention with the detail and particularity required by the Patent Laws, what is desired protected by Letters Patent is set forth in the following claims.

The invention claimed is:

1. A surgically-implantable breast prosthesis comprising: an enclosing shell having an exterior surface, an interior surface, and enclosing a lumen, said exterior surface defining an edge portion of said enclosing shell as viewed from a front direction, said edge portion separating front and rear portions of said enclosing shell, wherein the lumen enclosed by the shell is able to accommodate a fluid therein, and the front or front and rear portions of said enclosing shell being reinforced, thickened or stiffened on a central surface spaced from said edge portion.

2. A surgically-implantable breast prosthesis comprising: a first enclosing shell having an exterior surface, an interior surface, and enclosing a lumen, said exterior surface defining an edge portion of said first enclosing shell as viewed from a front direction, said edge portion separating front and rear portions of said first enclosing shell, wherein the lumen enclosed by the first shell is able to accommodate a first fluid therein; a second enclosing shell having an exterior surface, an interior surface, and enclosing a lumen, said exterior surface defining an edge portion of said second enclosing shell as viewed from a front direction, said edge portion separating front and rear portions of said second enclosing shell, wherein the lumen enclosed by the second shell is able to accommodate a second fluid therein; and the front or front and rear portions of said first and/or front or front and rear portions of said second enclosing shells being reinforced on a central surface spaced from said edge portion.
